(19) United States
(12) Patent Application Publication
(10) Pub. No.: US 2012/0330056 A1
(43) Pub. Date: Dec. 27, 2012

(54) PROCESS FOR PREPARING CHEMICAL COMPOUNDS OF INTEREST BY NUCLEOPHILIC AROMATIC SUBSTITUTION OF AROMATIC CARBOXYLIC ACID DERIVATIVES SUPPORTING AT LEAST ONE ELECTRO-ATTRACTIVE GROUP

(75) Inventors: Jacques Mortier, Ecommoy (FR); Anne-Sophie Castanet, Le Mans (FR); Arnaud Nourry, Le Mans (FR); Mickael Belaud-Rotureau, Le Mans (FR)

(73) Assignees: UNIVERSITE DU MAINE, LE MANS (FR); CENTRE NATIONAL DE LA RECHERCHE SCIENTIFIQUE, PARIS (FR)

(21) Appl. No.: 13/578,973

(22) PCT Filed: Feb. 18, 2011
(86) PCT No.: PCT/FR11/50349

§ 371 Date: Aug. 14, 2012

(30) Foreign Application Priority Data
Feb. 19, 2010 (FR) 1051226
Jun. 11, 2010 (FR) 1054645
Dec. 10, 2010 (FR) PCT/FR2010/052674

(51) Int. Cl. C07C 51/347; C07C 227/06; C07C 51/367

(52) U.S. Cl. 562/456; 562/493; 562/492; 562/469; 562/458

(57) ABSTRACT

A method for preparing aromatic carboxylic acid derivatives by nucleophilic aromatic substitution, involves reacting an aromatic carboxylic acid derivative supporting only one carboxyl function, or one of the salts thereof, the carboxylic acid derivative supporting, orthogonally to the carboxyl function, a leaving group which is a fluorine or chlorine atom or an alkoxy group, chiral or otherwise and, in the latter case, preferably a methoxy group; the carboxylic acid derivative being substituted by at least one electro-attractive group other than the leaving group, preferably by a fluorine atom, with a MNu reagent, wherein M is a metal and Nu an optionally chiral nucleophile, the nucleophilic aromatic substitution reaction being carried out without a catalyst and without a step of protecting/unprotecting the acid function of the initial compound, the method being selective in that the reaction leads to the formation of ketone derivatives in a very minority fashion during the reaction.

### FIELD OF THE INVENTION

This invention relates to the field of chemical synthesis, and in particular the invention proposes a new process enabling to perform a nucleophilic aromatic substitution on aromatic carboxylic acid derivatives bearing at least one electron withdrawing group other than the leaving group, in the absence of a catalyst and without a step of protection/deprotection of the acid function of the starting compound.

### PRIOR ART

Nucleophilic aromatic substitution is a reaction whose the interest is well known, and which is widely used in industry. However, it has disadvantages, which are widely reported, in particular the requirement to use catalysts, and the requirement to protect/deprotect the carboxyl function (CO₂H), necessary as a carbon anchoring point for subsequent chemical functionalization.

The use of catalysts is restrictive because they have to be trapped and removed at the end of the reaction. They are polluting residues and are also susceptible of leaving traces of heavy metals in the reaction products (see, for example, Königsberger et al, Organic Process Research & Development 2003, 7, 733-742, or Pink et al. Organic Process Research & Development 2008, 12, 589-595).

The need for protection/deprotection of the carboxyl function (CO₂H) is considered as a limiting requirement of nucleophilic substitution. It is indeed generally accepted that the CO₂H function reacts with organometallic compounds to lead to ketone derivatives, generally undesired (Jorgenson, M. J. Org. React. 1970, 18, 1. Ahn, T.; Cohen, T. Tetrahedron Lett. 1994, 35, 203). Therefore, the protection of the carboxylic function at the start of the nucleophilic substitution reaction appears to be an compulsory step. The protective groups used are generally sterically bulky and are considered to promote nucleophilic substitution.

The ability to overcome these requirements for catalysis and protection/deprotection is therefore a constant technical problem in the chemical and pharmaceutical industry.

In the application FR 1051226, the Applicant discloses a process of nucleophilic aromatic substitution on an industrial scale and with a high yield, and an optimized number of steps. In this process, the nucleophilic aromatic substitution reaction is performed on a carboxylic acid derivative or a salt thereof, said derivative being not substituted by an electron withdrawing group other than the leaving group.

The Applicant, in pursuing his work, observed surprisingly that the use of carboxylic acid derivatives substituted by at least one electron withdrawing group other than the leaving group, in particular difluorobenzoic acids, as starting compound, enabled him to avoid any nucleophilic attack on the carboxylate, nevertheless unprotected. As a consequence, ketone formation becomes very minor when the experimental conditions are well chosen and the ipso-substitution products of interest are predominantly obtained. In particular, the presence of a first fluorine atom in ortho position of the carboxyl function and a second fluorine atom in position 4 or 6 of the aromatic ring renders the carobxylate inert to nucleophilic attack. This invention therefore makes it possible to minimize the formation of by-products.

### General Description

Thus, the invention relates to a selective process for preparing aromatic carboxylic acid derivatives by nucleophilic aromatic substitution, wherein the following are reacted:
an aromatic carboxylic acid derivative bearing a carboxyl function and a single one, or a salt thereof, preferably a lithium salt, a sodium salt, a potassium salt or a zinc salt, preferably a benzoic acid derivative or a salt thereof,
   said carboxylic acid derivative has, in ortho position of the carboxyl function, a leaving group, which is a fluorine or chlorine atom or a chiral or non-chiral alkoxy group, and in this latter case, a methoxy group is preferred;
   said carboxylic acid derivative is substituted on a position of the ring that is not that occupied by the leaving group, by at least one electron withdrawing group, preferably a fluorine atom,
with a MNu reactant, wherein M is a metal and Nu is a chiral or non-chiral nucleophile,
given that:
   if the leaving group is a fluorine atom, and there is a bromine atom in para position, and the other positions are substituted by hydrogen atoms, then NuM is not iBuMgCl or NuMgBr where Nu is the ethyl or isobutyl or cyclopentenyl group,
   if the leaving group is a fluorine atom, and there is a halogen in the other ortho position, and there is a fluorine atom in para position as well as in the meta position adjacent to the leaving group and the other meta position is substituted by a hydrogen atom, then NuM is not an alkylating agent wherein Nu is C₁₋₆ alkyl,
   if the starting compound is 2,3,4,6-tetrafluorobenzoic acid, then NuM is not MeMgBr,
said nucleophilic aromatic substitution reaction is performed without catalyst and without step of protection/deprotection of the acid function of the starting compound,
this process being selective in that the reaction leads to the very minor formation of ketone derivatives during the reaction.

Preferably, the aromatic carboxylic acid derivative, starting product of the reaction, is a benzoic acid derivative of general formula (II):

wherein
   R1 is CO₂H,
   R2 is a fluorine or chlorine atom or a chiral or non-chiral alkoxy group, preferably OCH₃
   R3 is a hydrogen atom, an alkyl group, an alkoxy group, an aryl or an amine substituted or not by one or two alkyl groups or an electron withdrawing group, or R3 is a substituent capable of reacting in presence of a base and a metal to form MNu, or R3 may form a ring with R4,
   R4 is a hydrogen atom, an alkyl group, an alkoxy group, an aryl, or an amine substituted or not by one or two alkyl groups or an electron withdrawing group, or is a substituent capable of reacting in presence of a base and a metal to yield MNu, or R4 may form a ring with R3 or R5,
   R5 is a hydrogen atom, an alkyl group, an alkoxy group, an aryl, or an amine substituted or not by one or two alkyl groups or an electron withdrawing group, or is a substituent capable of reacting in presence of a base and a metal to yield MNu, or R5 may form a ring with R4 or R6,
   R6 is a hydrogen atom, an alkyl group, an alkoxy group, an aryl, or an amine substituted or not by one or two alkyl groups or an electron withdrawing group, or is a substituent capable of reacting in presence of a base and a metal to yield MNu, or R6 may form a ring with R5
given that at least one of R3, R4, R5 and R6 is an electron withdrawing group,
which is reacted with
a compound (III) of general formula NuM wherein Nu is a nucleophile, and M is a metal, preferably Li, Mg, Zn, Cu or an organomagnesium derivative MgX wherein X is a halogen atom or an alkoxy group, preferably OCH₃,
said nucleophilic aromatic substitution reaction is performed without catalyst and without step of protection/deprotection of the acid function of the compound (II),
to selectively obtain a compound of general formula (I), which corresponds to general formula (II) wherein at least R2 has been substituted by Nu, given that:
   if the leaving group is a fluorine atom, and the para position is substituted by a bromine atom and the other positions are substituted by hydrogen atoms, then NuM is not iBuMgC1 or NuMgBr wherein Nu is the ethyl or isobutyl or cyclopentenyl group,
   if the leaving group is a fluorine atom, and there is a halogen in the other ortho position, and there is a fluorine atom in para position as well as in the meta position adjacent to the leaving group, and the other meta position is occupied by a hydrogen atom, then NuM is not an alkylating agent wherein Nu is C₁₋₆ alkyl,
   if the starting product is 2,3,4,6-tetrafluorobenzoic acid, then NuM is not MeMgBr.

According to a preferred embodiment, at least one of R4 or R6 is an electron withdrawing group, and the other being as defined above, and in this embodiment
according to a first alternative, when R6 is an electron withdrawing group, and when R4 and R5 do not form a ring, then R3 and R4 may form together an aromatic ring or not, or a heterocycle, optionally substituted, in particular by a functional group
according to a second alternative, when R6 is an electron withdrawing group, and when R3 and R4 do not together form a ring, then R4 and R5 may form together an aromatic ring or not, or a heterocycle, optionally substituted, in particular by a functional group
according to a third alternative, when R4 is an electron withdrawing group, then R5 and R6 may form together an aromatic ring or not, or a heterocycle, optionally substituted, in particular by a functional group.

According to an embodiment, when R3 is a substituent capable of reacting in presence of a base and a metal to afford MNu, then the substitution of the leaving group R2 by NuM leads to an intramolecular reaction.

According to an embodiment, R4, R5 or R6 is a substituent capable of reacting in presence of a base and a metal to form MNu when one of the adjacent positions thereof is occupied by a substituent capable of acting as a leaving group, leading to an intramolecular reaction.

### Procedure

Advantageously, the reaction is performed between −78° C. and solvent reflux. Preferably, the reaction is performed in a polar aprotic solvent, preferably anhydrous THF (tetrahydrofuran) or diethyl ether, benzene, toluene or a hydrocarbon such as pentane, hexane, heptane or octane.

Advantageously, NuM compound is preferably added dropwise, at a temperature between −78° C. and solvent reflux.

Preferably, the solution is stirred, and then hydrolyzed with water. Advantageously, the hydrolysis is performed at low temperature. pH is adjusted to 1 with an aqueous chlorhydric acid solution (2N) and the solution is extracted with an appropriate solvent, for example ethyl acetate. The organic phase is then dried and concentrated under vacuum. The raw product is recrystallized or chromatographied.

According to an embodiment of the invention, at least one equivalent of NuM is used for one equivalent of starting aromatic carboxylic acid derivative. Advantageously, in addition to this equivalent, one equivalent of NuM is added per leaving group of the starting molecule to be substituted.

According to another embodiment of the invention, at least one equivalent of a metallic base, preferably butyllithium, sodium hydride, potassium hydride or lithium hydride is used for one equivalent of starting aromatic carboxylic acid derivative in order to form the metal salt corresponding to the acid function of the aromatic carboxylic acid derivative, and at least one equivalent of NuM is added per leaving group of the staring molecule to be substituted.

The reaction is selective because the ketone is formed in a very minor amount (<10%). Expected yields for the reaction process according to the invention are between 45 and 100%, preferably 45 to 90%, and more preferably 60 to 90%.

### Specific Cases

### Presence of an Asymmetric Carbon

According to a preferred embodiment, an asymmetric carbon is present on said aromatic carboxylic acid derivative, preferably on said benzoic acid derivative of general formula (II) and/or on the nucleophile, and the compound of general formula (I) obtained is asymmetric. Very advantageously, aromatic carboxylic acid derivative, preferably said benzoic acid derivative of general formula (II), has at least one chiral leaving group.

### Use of a Chiral Ligand

In a specific embodiment, a chiral ligand is added to the reaction mixture; this ligand is intended to provide chirality to the product (I) of the reaction of the invention.

According to the invention, said chiral ligand may be selected from chiral diamines, chiral diethers, chiral aminoethers, multi-point binding chiral aminoethers and bisoxazoline ligands. Examples of chiral ligands capable of being used are depicted in table 1.

TABLE 1

| | |
|---|---|
| Example of chiral diamine | |
| Example of chiral diether | |
| Example of chiral aminoether | |
| Example of multi-point binding chiral aminoether | |
| Example of bisoxazoline ligand | |

### Specific Cases Wherein R2 is a Fluorine or Chlorine Atom

According to a first embodiment, when R2 is a fluorine or chlorine atom, then Nu is not a substituted or non-substituted amine, in particular Nu is not an aniline derivative.

According to a second embodiment, when R2 is a fluorine or chlorine atom, then Nu is not a substituted or non-substituted amine.

According to a third embodiment, R2 is a fluorine or chlorine atom, and the nucleophile of the compound of general formula NuM is an aniline derivative. In this embodiment, according to a first aspect, compound NuM is obtained according to the synthesis routes described below, given that NuM is not the product of reaction between the nucleophile and a metallic base selected from lithium hydride, sodium hydride, potassium hydride, calcium hydride, lithium diisopropylamide, lithium amide, sodium amide, potassium amide, sodium methoxide, sodium ethoxide, potassium tert-butoxide, magnesium ethoxide and LiHMDS. In this embodiment, according to a second aspect, compound NuM is obtained by a reaction of nucleophile and butyllithium.

### Specific Cases of Difluorobenzoic Acids

According to a specific embodiment of the process of the invention, the compound of general formula (II) is such that:
R1 is CO₂H,
R2 and R6 are each independently a fluorine atom, and
R3, R4, R5 are each independently a hydrogen atom.

The reaction of this specific compound with a nucleophile NuM affords only the mono- or di-substituted product. The corresponding ketones are not formed and the carboxyl function does not undergo nucleophilic attacks.

Thus, the following mono-substituted product or a mixture of mono- and di-substituted products is obtained:

According to another specific embodiment of the process according to the invention, the compound of general formula (II) is such that:
R1 is CO₂H,
R2 and R4 are each independently a fluorine atom, and
R3, R5, R6 are each independently a hydrogen atom

The reaction of this specific compound with a nucleophile NuM produces the mono-substituted product only. The corresponding ketones are not formed and the carboxyl function does not undergo nucleophilic attacks.

The mono-substituted product or a mixture of mono- and di-substituted products is obtained.

Obtaining the NuM compound (III)

According to a first embodiment, compound NuM may be obtained by direct synthesis (Carey & Sundberg, Advanced Organic Chemistry, Part A Chapter 7, “Carbanions and Other Nucleophilic Carbon Species”, pp. 405-448).

According to a second embodiment, compound NuM may be obtained from lithium salts and anion radicals (T. Cohen et al. JACS 1980, 102, 1201; JACS 1984, 106, 3245; Acc. Chem. Res, 1989, 22, 52).

According to a third embodiment, compound NuM may be obtained by metal-halogen exchange (Parham, W. E.; Bradcher, C. K. Acc. Chem. Res. 1982, 15, 300-305).

According to a fourth embodiment, compound NuM may be obtained by directed metallation (V. Snieckus, Chem. Rev, 1990, 90, 879; JOC 1989, 54, 4372).

According to a preferred embodiment of the invention, compound NuM is obtained by reaction of the nucleophile and n-BuLi.

According to a preferred embodiment of the invention, compound NuM is obtained by reaction of the nucleophile and a base, in particular a metallic or an organometallic base. According to a first embodiment, the base is not LiNH₂. According to a second embodiment, the metallic base is not selected from the group consisting of lithium hydride, sodium hydride, potassium hydride, calcium hydride, lithium diisopropylamide, lithium amide, sodium amide, potassium amide, sodium methoxide, sodium ethoxide, potassium tert-butoxide, magnesium ethoxide, and LiHMDS. According to a third embodiment, the base is butyllithium, and in this embodiment, advantageously, compound NuM is obtained by reaction of nucleophile and n-BuLi. According to a fourth embodiment, the base is chiral and induces chirality to NuM.

Preferably, Nu is a nucleophile selected from those depicted in tables 2, 3 and 4.

TABLE 2

| Nu | M |
|---|---|
| Alkyl, preferably CH₃ or C₂H₅ | Li, Mg, Cu, Zn, or MgX wherein X is a |
| | halogen or an alkoxy |
| alkenyl, optionally substituted | Li, Mg, Cu, Zn, or MgX wherein X is a |
| | halogen or an alkoxy |
| Alkynyl optionally substituted | Li, Mg, Cu, Zn, or MgX wherein X is a |
| | halogen or an alkoxy |
| Aryl optionally substituted | Li, Mg, Cu, Zn, or MgX wherein X is a |
| | halogen or an alkoxy |
| s-Bu | Li, Mg, Cu, Zn, or MgX wherein X is a |
| | halogen or an alkoxy |
| t-Bu | Li, Mg, Cu, Zn, or MgX wherein X is a |
| | halogen or an alkoxy |
| n-Bu | Li, Mg, Cu, Zn, or MgX wherein X is a |
| | halogen or an alkoxy |
| 4-MeOC₆H₄ | Li, Mg, Cu, Zn, or MgX wherein X is a |
| | halogen or an alkoxy |
| 2-MeOC₆H₄ | Li, Mg, Cu, Zn, or MgX wherein X is a |
| | halogen or an alkoxy |
| 2,5-diMeC₆H₄ | Li, Mg, Cu, Zn, or MgX wherein X is a |
| | halogen or an alkoxy |
| 4-Me₂NC₆H₄ | Li, Mg, Cu, Zn, or MgX wherein X is a |
| | halogen or an alkoxy |
| | Li, Mg, Cu, Zn, or MgX wherein X is a halogen or an alkoxy |
| 2-MeC₆H₄ | Li, Mg, Cu, Zn, or MgX wherein X is a |
| | halogen or an alkoxy |
| | Li, Mg, Cu, Zn, or MgX wherein X is a halogen or an alkoxy |
| | Li, Mg, Cu, Zn, or MgX wherein X is a halogen or an alkoxy |
| | Li, Mg, Cu, Zn, or MgX wherein X is a halogen or an alkoxy |
| | Li, Mg, Cu, Zn, or MgX wherein X is a halogen or an alkoxy |
| | Li, Mg, Cu, Zn, or MgX wherein X is a halogen or an alkoxy |
| wherein Y is O, N or S | Li, Mg, Cu, Zn, or MgX wherein X is a halogen or an alkoxy |
| wherein Y is O, N or S | Li, Mg, Cu, Zn, or MgX wherein X is a halogen or an alkoxy |
| P(Aryl)₂ | Li, Mg, Cu, Zn, or MgX wherein X is a |
| | halogen or an alkoxy |
| PArylAlkyl | Li, Mg, Cu, Zn, or MgX wherein X is a |
| | halogen or an alkoxy |
| O(C₁₋₆alkyl) | Li, Mg, Cu, Zn, or MgX wherein X is a |
| | halogen or an alkoxy |
| S(C₁₋₆alkyl) | Li, Mg, Cu, Zn, or MgX wherein X is a |
| | halogen or an alkoxy |
| wherein R¹⁸ is a hydrogen atom, an alkyl group, an alkoxy group, an aryl or an amine substituted or not by one or two C₁₋₁₂alkyl groups | Li, Mg, Cu, Zn, or MgX wherein X is a halogen or an alkoxy |

TABLE 3

| Nu | M |
|---|---|
| N(C₁₋₆alkyl)₂ | Li, Mg, Cu, Zn, or MgX wherein X is a |
| | halogen or an alkoxy |
| NH(C₁₋₆alkyl), in | Li, Mg, Cu, Zn, or MgX wherein X is a |
| particular NH(tBu) | halogen or an alkoxy |
| NEt₂ | Li, Mg, Cu, Zn, or MgX wherein X is a |
| | halogen or an alkoxy |
| | Li, Mg, Cu, Zn, or MgX wherein X is a halogen or an alkoxy |
| N(iPr)₂ | Li, Mg, Cu, Zn, or MgX wherein X is a |
| | halogen or an alkoxy |
| | Li, Mg, Cu, Zn, or MgX wherein X is a halogen or an alkoxy |
| | Li, Mg, Cu, Zn, or MgX wherein X is a halogen or an alkoxy |
| | Li, Mg, Cu, Zn, or MgX wherein X is a halogen or an alkoxy |
| N(CH₂CH₂)₂NMe | Li, Mg, Cu, Zn, or MgX wherein X is a |
| | halogen or an alkoxy y |
| NMeBn | Li, Mg, Cu, Zn, or MgX wherein X is a |
| | halogen or an alkoxy |
| NBn₂ | Li, Mg, Cu, Zn, or MgX wherein X is a |
| | halogen or an alkoxy |
| NMePh | Li, Mg, Cu, Zn, or MgX wherein X is a |
| | halogen or an alkoxy |
| NHt-Bu | Li, Mg, Cu, Zn, or MgX wherein X is a |
| | halogen or an alkoxy |
| NPh₂ | Li, Mg, Cu, Zn, or MgX wherein X is a |
| | halogen or an alkoxy |

According to a first preferred embodiment of the invention, in tables 2 and 3, M is Li or Mg.

According to a preferred embodiment, M is Li, Mg, Cu, Zn, or MgX wherein X is a halogen or an alkoxy and Nu is N(C₁₋₆alkyl)₂, NH(C₁₋₆alkyl), NEt₂, N(CH₂CH₂)₂NMe, NMeBn, NBn₂, NMePh, NHt-Bu or NPh₂.

Advantageously, in tables 2 and 3, when M is MgX and X is a halogen, then the halogen is selected from F, Br, Cl. Advantageously, when M is MgX and X is an alkoxy, then the alkoxy is OCH₃ or OC₂H₅. According to a preferred embodiment of the invention, M is MgBr or MgOCH₃.

The preferred chiral NuM compounds according to the invention are exemplified in table 4 below.

TABLE 4

| Nu | M |
|---|---|
| | Li, Mg |
| | Li, Mg |
| | Li, Mg |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| | Li, Mg, Cu, Zn |
| wherein Y is O, S or N | Li, Mg |
| wherein Y is O, S or N | Li, Mg |
| wherein Y is O, S or N | Li, Mg |
| wherein Y is O, S or N | Li, Mg |
| NR¹¹R¹²* wherein R¹¹ and R¹² are each | Li, Mg |
| independently a hydrogen atom, an alkyl | |
| group, an alkoxy group, an aryl, or an | |
| amine substituted or not by one or two | |
| C₁₋₁₂alkyl groups. | |
| SiR¹³R¹⁴R¹⁵* wherein R13, R14 and R15 | Li, Mg |
| are each independently a hydrogen atom, | |
| an alkyl group, an alkoxy group, an aryl, or | |
| an amine substituted or not by one or | |
| two C₁₋₁₂alkyl groups. | |
| OR¹⁶* wherein R¹⁶ is a hydrogen atom, an | Li, Mg |
| alkyl group, an alkoxy group, an aryl, or | |
| an amine substituted or not by one or two | |
| C₁₋₁₂alkyl groups. | |
| SR¹⁷* wherein R¹⁷ is a hydrogen atom, an | Li, Mg |
| alkyl group, an alkoxy group, an aryl, or | |
| an amine substituted or not by one or two | |
| C₁₋₁₂alkyl groups. | |

*chiral element

According to a specific embodiment of the invention, each non-substituted position of an aromatic ring depicted in one of tables 2 to 4 may be substituted by a hydrogen atom, an alkyl group, an alkoxy group, an aryl, or an amine substituted or not by one or two C₁₋₁₂alkyl groups.

Preferably, M is Li or MgBr; preferably, Nu is n-Bu, s-Bu, t-Bu, methyl, phenyl, 2-MeC₆H₄, 2-MeOC₆H₄, 4-MeC₆H₄, 4-MeOC₆H₄ or naphthalene.

The preferred NuM compounds are n-Buli, s-Buli, t-Buli, MeLi, PhLi, PhMgBr, 2-MeC₆H₄Li, 2-MeOC₆H₄Li, 4-MeC₆H₄Li, 4-MeOC₆H₄Li, 1-LiNaphthalene, 2-LiNaphthalene.

### DEFINITIONS

In the sense of this invention, the term “aryl” means a mono- or polycyclic system of 5 to 20, and preferably 6 to 12, carbon atoms having one or more aromatic rings (when there are two rings, it is called a biaryl) among which it is possible to cite the phenyl group, the biphenyl group, the 1-naphthyl group, the 2-naphthyl group, the tetrahydronaphthyl group, the indanyl group and the binaphthyl group. The term aryl also means any aromatic ring including at least one heteroatom chosen from an oxygen, nitrogen or sulfur atom. The aryl group may be substituted by 1 to 3 substituents chosen independently of one another, among hydroxyl group; linear or branched alkyl group comprising 1, 2, 3, 4, 5 or 6 carbon atoms, in particular methyl, ethyl, propyl, butyl; alkoxy group or halogen atom, in particular bromine, chlorine and iodine.

The term “catalyst” refers to any product involved in the reaction for increasing the speed of said reaction, but regenerated or removed during or at the end of the reaction.

By “protecting the carboxyl function (CO₂H)”, we mean adding to said function a group destroying the reactivity of the carboxyl function with regard to the nucleophiles; this group may be oxazoline; numerous chemical groups other than the oxazoline function have been used to protect the CO₂H function: 2,6-di-tert-butyl-4-methoxyphenylic ester (Hattori, T.; Satoh, T.; Miyano, S. Synthesis 1996, 514. Koshiishi, E.; Hattori, T.; Ichihara, N.; Miyano, S. J. Chem. Soc., Perkin Trans. 1 2002, 377), amide (Kim, D.; Wang, L.; Hale, J. J.; Lynch, C. L.; Budhu, R. J.; MacCoss, M.; Mills, S. G.; Malkowitz, L.; Gould, S. L.; DeMartino, J. A.; Springer, M. S.; Hazuda, D.; Miller, M.; Kessler, J.; Hrin, R. C.; Carver, G.; Carella, A.; Henry, K.; Lineberger, J.; Schleif, W. A.; Emini, E. A. Bioorg. Med. Chem. Lett. 2005, 15(8), 2129), alkylamide (Guo, Z.; Schultz, A. G. Tetrahedron Lett. 2001, 42(9), 1603), dialkylamides (Hoarau, C.; Couture, A.; Deniau, E.; Grandclaudon, P. Synthesis 2000), 1-imidazolyles (Figge, A.; Altenbach, H. J.; Brauer, D. J.; Tielmann, P. Tetrahedron: Asymmetry 2002, 13(2), 137), 2-oxazolyles (Cram, D. J.; Bryant, J. A.; Doxsee, K. M. Chem. Lett. 1987, 19), 2-thiazolyles, etc.

By “leaving group” we mean a group that leads the two electrons of the sigma bond binding it with the aromatic carbon atom during the substitution reaction with the nucleophile; according to the invention, the leaving group may be chiral or non-chiral; according to a preferred embodiment of the invention, the leaving group is chiral; according to the invention, the leaving group may be electron withdrawing or non-electron withdrawing.

By “alkyl”, we mean any saturated linear or branched hydrocarbon chain, with 1 to 12 carbon atoms, preferably 1 to 6 carbon atoms, and more preferably methyl, ethyl, propyl, isopropyl, n-butyl, sec-butyl, isobutyl and tert-butyl.

By “alkoxy”, we mean any O-alkyl or O-aryl group.

By “alkenyl”, we mean any linear or branched hydrocarbon chain having at least one double bond, of 2 to 12 carbon atoms, and preferably 2 to 6 carbon atoms.

By “alkynyl”, we mean any linear or branched hydrocarbon chain having at least one triple bond, of 2 to 12 carbon atoms, and preferably 2 to 6 carbon atoms.

By “amine”, we mean any compound derived from ammonia NH₃ by substitution of one or more hydrogen atoms with an organic radical. According to the invention, a preferred amine is an aniline derivative.

By “functional group”, we mean a sub-molecular structure including an assembly of atoms conferring a reactivity specific to the molecule that contains it, for example an oxy, carbonyl, carboxy, sulfonyl group, and so on.

By “nucleophile”, we mean an acyclic or cyclic compound, of which the characteristic is to include at least one atom with a free electron pair, charged or not. According to a preferred embodiment of the invention, we mean by “nucleophile” an acyclic or cyclic compound of which the characteristic is to include at least one atom with a charged free electron pair, preferably negatively charged.

By “nucleophile that may be chiral”, we mean a nucleophile with at least one asymmetric carbon.

By “electron withdrawing group” we mean a functional group having the ability to attract electrons, in particular if it is a substitutent of an aromatic group, for example a group in particular of the NO₂, CN, halogen, CO₂R, CONR₂, CH═NR, (C═S)OR, (C═O)SR, CS₂R, SO₂R, SO₂NR₂, SO₃R, P(O)(OR)₂, P(O)(R)₂, or B(OR)₃ type wherein R is an alkyl, an aryl or a hydrogen atom. Amines and alkoxy groups are not electron withdrawing groups.

By “heterocycle”, we mean a ring with 5- or 6-membered ring containing 1 to 2 heteroatoms selected from O, S, N, optionally substituted with an alkyl.

By “MNu”, we mean a reactant wherein M is a metal and Nu is an independent nucleophile or a substituent of the aromatic ring of the benzoic acid derivative of general formula (II), said substituent being capable of reacting in presence of a base and a metal to form MNu. When Nu is a substituent of the aromatic ring of (II), the nucleophilic aromatic substitution reaction occurs intramolecularly between the MNu function formed on the substituent and the leaving group in ortho position to carboxylic acid function.

The invention may be better understood in view of the following examples, which illustrate the process according to the invention in a non-limiting manner.

### EXAMPLES

All of the reactions are done under inert atmosphere with anhydrous solvents (Gordon, J. A.; Ford, R. A. The Chemist's Companion, Wiley J. and Sons, New York, 1972). The THF is distilled by means of an anhydrous THF GTS100 station (Glass Technology). Alkyllithium derivatives are periodically titrated with N-benzylbenzamide (Burchat, A. F.; Chong, J. M.; Nielsen, N. J. Organomet. Chem. 1997, 542, 281)

S-butyllithium (1.4 M in solution in cyclohexane), n-butyllithium (1.6 M in solution in hexane), t-butyllithium (1.7 M in solution in pentane) and phenyllithium (1.8 M in solution in dibutylether) are sold by Acros Chemicals and Aldrich Chemical Company.

Nuclear magnetic resonance spectra of the proton ¹H (400 MHz or 200 MHz) and of the carbon ¹³C (50 MHz or 100.6 MHz) were recorded on a Bruker AC 400 or DPX 200 apparatus. The chemical shifts 6 are expressed in parts per million (ppm).

Tetramethylsilane (TMS) is used as an internal reference when CDCl₃ is used as a solvent. In the case of acetone-d₆ and DMSO d₆, chemical shifts are given with respect to the signal of the solvent. Coupling constants are expressed in Hertz (Hz). The following abbreviations are used to describe the NMR spectra: s (singlet), d (doublet), dd (double doublet), t (triplet), q (quadruplet), m (multiplet), sept (septuplet)

The mass spectra were recorded in chemical impact mode or in field ionization mode on a high-resolution spectrometer (GCT First High-Resolution Micromass). The precision obtained for the precise mass measurements is four digits.

Elemental analyses were performed by the microanalysis center of ICSN of-Gif sur Yvette. Infrared spectra were recorded on a Nicolet® Avatar® 370 DTGS spectrometer. Melting points were measured on a Biichi Melting Point B-540 apparatus.

### Example 1

### Preparation of 2-n-butyl-6-fluorobenzoic Acid

n-BuLi (6.9 mL, 11 mmol, 1.6 M in solution in hexane) is added at −78° C. to a solution of 2,6-difluorobenzoic acid (791 mg, 5 mmol) in anhydrous THF (30 mL). The reaction mixture is stirred at this temperature for 2 h, and then iodomethane (1.25 mL, 12 mmol) is added. The solution is hydrolyzed at room temperature with water (20 mL) and the two phases are separated. The aqueous phase is washed with ethyl acetate (3×40 mL). The aqueous phase is then acidified to a pH of 1 and extracted with ethyl acetate (3×40 mL). The combined organic phases are dried over MgSO4 and concentrated under vacuum. The residue is purified by chromatography on silica gel (cyclohexane:ethyl acetate 95:5) to afford 2-butyl-6-fluorobenzoic acid (425 mg, 2.17 mmol, 43%) as a yellow oil Addition of iodomethane before hydrolysis does not modify the outcome of the reaction. ¹H NMR (400 MHz, CDCl₃) δ: 11.04 (s large, 1H), 7.35 (td, JHF=5.7 Hz, J=8.0 Hz, 1H, H5), 7.05 (d, J=7.6 Hz, 1H, H4), 6.97 (dd, J=8.2 Hz, JHF=9.6 Hz, 1H, H6), 2.81 (t, J=7.8 Hz, 2H), 1.62 (m, 2H) 1.38 (m, 2H), 0.93 (t, J=7.3 Hz, 3H). ¹³C NMR (100 MHz, CDCl₃) δ: 171.6, 160.3 (d, J=253 Hz), 144.2 (d, J=1.3 Hz), 131.9 (d, J=9.2 Hz), 120.0 (d, J=14.3 Hz), 125.5 (d, J=3.2 Hz), 113.4 (d, J=21.8 Hz), 33.5, 33.2, 22.5, 13.8. IR (ATR, cm⁻¹): 2960, 2873, 2662, 2873, 1704, 1615, 1576, 1467, 1405, 1293, 1125, 805, 775. HRMS [M+NH₄]⁺ calculated for C₁₁H₁₇NO₂F: 214.1243, measured: 214.1246.

### Example 2

### Preparation of 2,6-di-sec-butylbenzoic Acid

This compound is prepared from 2,6-difluorobenzoic acid (791 mg, 5 mmol) and s-BuLi (10.7 mL, 15.0 mmol, 1.4 M in solution in cyclohexane) according to the procedure of example 1. The reaction mixture is stirred at 0° C. during 4 h. Purification by recrystallization (cyclohexane/ethyl acetate) yielded 2,6 di-sec-butylbenzoic acid (650 mg, 2.77 mmol, 55%) as a white solid (mp 125-126° C.). Addition of iodomethane before hydrolysis does not modify the outcome of the reaction. ¹H NMR (400 MHz, CDCl₃) δ: 7.36 (t, J=7.8 Hz, 1H), 7.13 (d, J=7.8 Hz, 2H), 2.73 (sext, J=7.0 Hz, 2H), 1.75-1.55 (m, 4H), 1.27 (dd, J=1.6 Hz, J=6.8 Hz, 6H), 0.85 (t, J=7.4 Hz, 6H). ¹³C NMR (100 MHz, CDCl₃) δ: 176.2, 143.2, 133.4, 129.5, 122.8, 38.7, 30.9, 22.0, 12.1. IR (ATR, cm⁻¹): 2955, 2925, 2864, 1705, 1594, 1585, 1456, 1390, 1379, 1260, 1134, 1003, 908, 803, 764, 699, 609. HRMS [M+NH₄]⁺ calculated for C₁₅H₂₆NO₂: 252.1964, measured: 252.1963.

### Example 3

### Preparation of 3-fluorobiphenyl-2-carboxylic Acid

This compound is prepared from 2,6-difluorobenzoic acid (474 mg, 3 mmol) and PhLi (4.55 mL, 6.6 mmol, 1.45 M in solution in di-n-butyl ether) according to the general procedure. The reaction mixture is stirred at −30° C. during 2 h. The compound is recovered and purified by column chromatography on silica gel (cyclohexane:ethyl acetate 95:5 to 90:10) affording 3-fluorobiphenyl-2-carboxylic acid (185 mg, 0.856 mmol, 29%) as a yellow solid (mp 122.5-125° C.). ¹H NMR (200 MHz, CDCl₃) δ: 7.53-7.40 (m, 6H), 7.22-7.09 (m, 2H). ¹³C NMR (50 MHz, CDCl₃) δ: 171.1, 159.8 (d, J=252.6 Hz), 142.8 (d, J=2.4 Hz), 139.0 (d, J=2.3 Hz), 131.7 (d, J=9.1 Hz), 128.5 (2*C), 128.2 (2*C), 128.1, 125.7 (d, J=3.2 Hz), 120.3 (d, J=15.7 Hz), 114.7 (d, J=21.6 Hz). IR (ATR, cm⁻¹): 2860, 2654, 1690, 1612, 1567, 1460, 1401, 1293, 1267, 1238, 1127, 1097, 897, 803, 771, 702, 549. HRMS [M]⁺ calculated for C₁₃H₉FO₂: 216.0587, measured: 216.0587.

### Example 4

### Preparation of 3-fluoro-4-methoxy-biphenyl-2-carboxylic Acid

n-BuLi (7.9 mL, 11 mmol, 1.39 M in solution in hexane) is added at −78° C. dropwise to a 1-bromo-4-methoxybenzene solution (2.057 g, 1.40 mL, 11 mmol) in anhydrous THF (20 mL). The reaction mixture is stirred at this temperature for 1 h, then warmed up to −50° C. and 2,6-difluorobenzoic acid (791 mg, 5 mmol) in solution in anhydrous THF is then added. The reaction mixture is warmed up to −30° C. and is stirred at this temperature during 2 h. The solution is hydrolyzed at room temperature with water (25 mL) and the two phases are separated. The aqueous phase is washed with ethyl acetate (3×40 mL). The aqueous phase is then acidified to a pH of 1 and extracted with ethyl acetate (3×40 mL). The combined organic phases are dried over MgSO4 and concentrated under vacuum. The residue is purified by chromatography on silica gel (cyclohexane:ethyl acetate 95:5 to 8:2). 3-fluoro-4-methoxybiphenyl-2-carboxylic acid is isolated (803 mg, 3.26 mmol, 65%) as a colorless oil. ¹H NMR (200 MHz, CDCl₃) δ: 7.50-7.30 (m, 3H), 7.20-7.06 (m, 2H), 6.97-6.90 (m, 2H), 3.84 (s, 3H). ¹³C NMR (50 MHz, CDCl₃) δ: 171.1, 159.8 (d, J=252.1 Hz), 159.6, 142.4 (d, J=2.5 Hz), 131.6 (d, J=9.2 Hz), 131.4 (d, J=2.4 Hz), 129.4 (2*C), 125.7 (d, J=3.1 Hz), 120.3 (d, J=15.7 Hz), 114.2 (d, J=21.5 Hz), 114.0 (2*C), 55.2. IR (ATR, cm⁻¹): 1703, 1698, 1610, 1514, 1462, 1455, 1288, 1236, 1178, 1094, 1029, 896, 806, 781, 692, 587. HRMS [M+H]⁺ calculated for C₁₄H₁₂FO₃: 247.0770, measured: 247.0780.

### Example 5

### Preparation of 2,6-bis-(diethylamino)benzoic Acid

2,6-difluorobenzoic acid (474 mg; 3 mmol) in solution in anhydrous THF (10 mL) is added dropwise at −30° C. to a lithium diethylamide solution (15 mmol, prepared according to the general procedure in 30 mL of THF). The reaction mixture is stirred at −30° C. during 1 h and then 3 h at 0° C. The reaction mixture is hydrolyzed at room temperature with distilled water (20 mL) and the two phases are separated. The aqueous phase (AQ-1) is extracted with ethyl acetate (3*20 mL) and the combined organic phases (ORGA1) are dried over MgSO₄. The ORGA1 phase contains predominantly to the carboxylate derived from the 2,6-bis(diethylamino)benzoic acid, 10 mL of a 1N aqueous NaOH solution is added in order to purify it and the reaction mixture is concentrated under reduced pressure. After acidification at pH=7 (with a solution of HCl 10%) and extraction with AcOEt, pure 2,6-bis(diethylamino)benzoic acid is isolated (180 mg; 0.69 mmol) as a white solid. The aqueous phase AQ-1 is then acidified with an HCl solution (10%) to pH=7 and extracted with dichloromethane (3*20 mL). The combined organic phases (ORGA2) are dried over MgSO₄. The ORGA2 phase contains pure 2,6-bis(diethylamino)benzoic acid (240 mg, 0.92 mmol). (overall yield: 420 mg, 53%).

According to the same procedure, but using 2,6-dimethoxybenzoic acid (546 mg; 3 mmol) as the starting material, 2,6-bis(diethylamino)benzoic acid is isolated with a 53% yield (420 mg). mp=112-114° C. ¹H NMR (CDCl₃; 200 MHz) δ: 7.38 (t; J=8.0 Hz, 1H), 6.90 (d; J=8.0 Hz; 2H), 3.21 (q; J=7.2 Hz; 8H), 1.11 (t; J=7.2 Hz; 12H). NMR ¹³C(CDCl₃; 100 MHz): 167.1; 150.7; 131.3; 119.6; 115.6; 48.7; 11.9. IR (ATR, cm⁻¹): 3430; 2671; 2612; 2072; 1582; 1459; 1368; 1262. HRMS m/z calculated for C₁₅H₂₅N₂O₂ ([M]⁺): 265.1871 found 265.1909.

### Example 6

### Preparation of 2-(N-methyl-N-phenyl)-6-fluorobenzoic Acid

2,6-difluorobenzoic acid (474 mg; 3 mmol) in solution in anhydrous THF (10 mL) is added dropwise at room temperature to a lithium (N-methyl-N-phenyl)amide solution (15 mmol, prepared according to the general procedure in 30 mL of THF). The solution is stirred at room temperature during 1 h then overnight at 60° C. The reaction mixture is hydrolyzed at room temperature with distilled water (20 mL) and the two phases are separated. The aqueous phase (AQ-1) is extracted with ethyl acetate (3*20 mL) then acidified with an HCl solution (10%) to pH=7 and extracted with dichloromethane (3*20 mL). The combined organic phases (ORGA2) are dried over MgSO₄. The ORGA2 phase contains pure 2-(N-methyl-N-phenyl)-6-fluorobenzoic acid (190 mg, 0.92 mmol). After acidification at pH=1 (with HCl 10%), the residual aqueous phase is extracted with dichloromethane. The resulting organic phase (ORGA3) is dried over MgSO₄. It contains protonated 2-fluoro-6-(N-methyl-N-phenyl)benzoic acid. 10 mL of a 1N aqueous NaOH solution are added in order to purify it and the reaction mixture is concentrated under reduced pressure. After acidification at pH=7 (with HCl 10%) and extraction with AcOEt, pure 2-(N-methyl-N-phenyl)-6-fluorobenzoic acid is isolated as a dark beige solid (340 mg). (overall yield: 530 mg, 72%). mp=120-122° C. ¹H NMR (CDCl₃; 200 MHz): 7.46 (d; J_{H,H}=8 Hz; J_{H,F}=6 Hz; 1H), 7.24 (dd; J=8.8 Hz; J=7.2 Hz; 2H); 7.06 (dd; J_{H,H}=8.8 Hz; J_{H,F}=9.6 Hz; 1H); 6.98 (d; J=8 Hz; 1H); 6.94 (t; J=7.2 Hz; 1H); 6.82 (d; J=8.8 Hz; 2H); 3.25 (s; 3H). NMR ¹³C (CDCl₃; 100 MHz): 166.0; 160.5 (J=260 Hz); 149.0; 148.3; 133.6 (d, J=10 Hz); 129.5; 123.7; 122.8; 121.4; 117.5; 114.1 (d, J=22 Hz); 41.4. NMR ¹⁹F (CDCl₃, 376 MHz)=−111.0. IR (ATR, cm⁻¹): 3063; 1705; 1613; 1495; 1350; 1161; 1209; 995; 825; 756; 694; 608.

### Example 7

### Preparation of 2,6-di-s-butylbenzoic acid

s-butyllithium (1.25 M in cyclohexane, 12 mL, 15 mmol) is added at 0° C. to 2,6-difluorobenzoic acid (474 mg, 3 mmol) in solution in anhydrous THF (20 mL). After 4 h of stirring at 0° C., the reaction mixture is hydrolyzed with distilled water (20 mL) and the aqueous phase is extracted with ethyl acetate (3*20 mL). The combined organic phases are dried over MgSO₄, filtered and concentrated under reduced pressure. After recrystallization (cyclohexane/ethyl acetate), 2,6-di-s-butylbenzoic acid is isolated as a white solid (650 mg, 56%). mp=125-126° C. ¹H NMR (CDCl₃; 200 MHz): 7.35 (t; J=7.8 Hz; 1H), 7.25 (d; J=7.8 Hz; 2H), 2.72 (m; 1H), 1.68 (m; 2H), 1.26 (d; J=7.0 Hz; 3H), 0.85 (t; J=7.4 Hz; 3H). ¹³C NMR (CDCl₃; 100 MHz): 176.5; 143.5; 133.0; 129.0; 122.5; 39.4; 31.5; 22.5; 12.0. IR (ATR, cm⁻¹): 2954; 2925; 2863; 1704; 1594; 1584; 1456; 1390; 1379; 1260; 1234; 1134.

### Example 8

### Preparation of 2-n-butyl-6-fluorobenzoic Acid

n-butyllithium (1.55 M in cyclohexane, 7.1 mL, 11 mmol) is added at 0° C. to 2,6-difluorobenzoic acid (790 mg, 5 mmol) in solution in anhydrous THF (30 mL). After stirring 2 h at 0° C., the reaction mixture is hydrolyzed with distilled water (30 mL). The aqueous phase is extracted with ethyl acetate (3*30 mL), acidified to pH=1 with the addition of HCl (10%) then extracted with ethyl acetate. The combined organic phases are dried over MgSO₄, filtered and concentrated under reduced pressure. After recrystallization (cyclohexane/ethyl acetate), 2-fluoro-6-n-butylbenzoic acid is isolated as a pale yellow solid (560 mg, 57%). ¹H NMR (CDCl₃; 200 MHz): 7.34 (dd; J_{H,H}=8.2 Hz; J_{H,F}=5.6 Hz; 1H), 7.04 (d; J=8.2 Hz; 1H), 6.96 (dd; J_{H,H}=8.2 Hz; J_{H,F}=9.6 Hz; 1H), 2.81 (t; J=7.6 Hz; 2H), 1.68 (m; 2H), 1.39 (m; 2H), 0.91 (t; J=7.6 Hz; 3H). ¹³C NMR (CDCl₃; 100 MHz): 172.1, 160.0 (d; J=250 Hz), 144.3; 132.0 (d; J=10 Hz); 131.2; 125.5 (d; J=14 Hz); 120.0 (d; J=21 Hz); 113.6; 33.6; 22.5; 13.8. IR (ATR, cm⁻¹): 2960; 2873; 2662; 1704; 1615; 1576; 1466; 1405; 1293; 1125; 805; 774.8.

1. Process for preparing aromatic carboxylic acid derivatives by nucleophilic aromatic substitution, wherein the following are reacted:
an aromatic carboxylic acid derivative bearing a carboxyl function and a single one, or a salt thereof, preferably a lithium, sodium, potassium salt or a zinc salt, preferably a benzoic acid derivative or a salt thereof,
said carboxylic acid derivative bearing, in ortho position of the carboxyl function, a leaving group, which is preferably a fluorine or chlorine atom or a chiral or non-chiral alkoxy group, and in this latter case, a methoxy group is preferred;
said carboxylic acid derivative being substituted by at least one electron withdrawing group other than the leaving group, preferably a fluorine atom,
with a MNu reactant, wherein M is a metal and Nu is a chiral or non-chiral nucleophile,
given that:
if the leaving group is a fluorine atom, and a bromine atom is in para position and the other positions are substituted by hydrogen atoms, then NuM is not iBuMgCl or NuMgBr where Nu is the ethyl or isobutyl or cyclopentenyl group,
if the leaving group is a fluorine atom, and there is a halogen in the other ortho position, and there is a fluorine atom in para position as well as in meta position adjacent to the leaving group and there is a hydrogen atom in the other meta position, then NuM is not an alkylating agent wherein Nu is C₁₋₆ alkyl,
if the starting compound is 2,3,4,6-tetrafluorobenzoic acid, then NuM is not MeMgBr,
said nucleophilic aromatic substitution reaction being performed without catalyst and without step of protection/deprotection of the acid function of the starting compound,
this process being selective in that the reaction leads to the very minor formation of ketone derivatives during the reaction.

2. Process according to claim 1, characterized in that said carboxylic acid derivative, starting compound of the reaction, is a benzoic acid derivative of general formula (II)
wherein
R1 is CO₂H,
R2 is a fluorine or chlorine atom or a chiral or non-chiral alkoxy group, preferably OCH₂,
R3 is a hydrogen atom, an alkyl group, and alkoxy group, an aryl or an amine substituted or not by one or two alkyl groups or an electron withdrawing group, or R3 is a substituent capable of reacting in presence of a base and a metal to form MNu, or R3 may form a ring with R4,
R4 is a hydrogen atom, an alkyl group, an alkoxy group, an aryl, or an amine substituted or not by one or two alkyl groups or an electron withdrawing group, or is a substituent capable of reacting in presence of a base and a metal to form MNu, or R4 may form a ring with R3 or R5,
R5 is a hydrogen atom, an alkyl group, an alkoxy group, an aryl, or an amine substituted or not by one or two alkyl groups or an electron withdrawing group, or is a substituent capable of reacting in presence of a base and a metal to form MNu, or R5 may form a ring with R4 or R6,
R6 is a hydrogen atom, an alkyl group, an alkoxy group, an aryl, or an amine substituted or not by one or two alkyl groups or an electron withdrawing group, or is a substituent capable of reacting in presence of a base and a metal to form MNu, or R6 may form a ring with R5
given that at least one of R3, R4, R5 and R6 is an electron withdrawing group,
which is reacted with
a compound (III) of general formula NuM wherein Nu is a nucleophile, and M is a metal, preferably Li, Mg, Zn, Cu or an organomagnesium derivative MgX wherein X is a halogen atom or an alkoxy group, preferably OCH₃,
said nucleophilic aromatic substitution reaction is performed without catalyst and without a step of protection/deprotection of the acid function of the compound (II),
to selectively obtain a compound of general formula (I), which corresponds to general formula (II) wherein at least R2 has been substituted by Nu,
given that:
if the leaving group is a fluorine atom, and a bromine atom is in para position and the other positions are substituted by hydrogen atoms, then NuM is not iBuMgCl or NuMgBr where Nu is ethyl or isobutyl or cyclopentenyl group,
if the leaving group is a fluorine atom, and there is a halogen on the other ortho position, and there is a fluorine atom in para position as well as meta position adjacent to the leaving group and there is a hydrogen atom on the other meta position, then NuM is not an alkylating agent wherein Nu is C₁₋₆ alkyl,
if the starting compound is 2,3,4,6-tetrafluorobenzoic acid, then NuM is not MeMgB.

3. Process according to claim 1, wherein NuM is such that M is Li, Mg, Cu, Zn, or MgX wherein X is halogen or alkoxy, and Nu is as described below:
```
Nu
Alkyl, preferably CH₃ or
C₂H₅
Alkenyl, optionally
substituted
Alkynyl optionally
substituted
Aryl optionally
substituted
s-Bu
t-Bu
n-Bu
4-MeOC₆H₄
2-MeOC₆H₄
2,5-diMeC₆H₄
4-Me₂NC₆H₄

2-MeC₆H₄





  wherein Y is O, N or S
  wherein Y is O, N or S
P(Aryl)₂,
PArylAlkyl
O(C₁₋₆alkyl)
S(C₁₋₆alkyl)
  wherein R¹⁸ is a hydrogen atom, an alkyl group, an alkoxy group, an aryl, or an amine substituted or not by one or two C₁₋₁₂alkyl groups
```

4. Process according to claim 1, wherein NuM is such that M is Li, Mg, Cu, Zn, or MgX wherein X is a halogen or an alkoxy and Nu is N(C₁₋₆alkyl)₂, NH(C₁₋₆alkyl), NEt₂, N(CH₂CH₂)₂NMe, NMeBn, NBn₂, NMePh, NHt-Bu NPh₂.

5. Process according to claim 1, wherein NuM is such that M is Li, Mg, Cu, Zn, or MgX wherein X is halogen or alkoxy, and Nu is as described below:
Nu
N(C₁₋₆alkyl)₂
NH(C₁₋₆alkyl),
in particular
NH(tBu)
NEt₂
N(iPr)₂
N(CH₂CH₂)₂NMe
NMeBn
NBn₂
NMePh
NHt-Bu
NPh₂

6. Process according to claim 1, wherein NuM is such that M is Li, Mg, and Nu is as described below:
```
Nu














  wherein Y is O, S or N
  wherein Y is O, S or N
  wherein Y is O, S or N
  wherein Y is O, S or N
NR¹¹R¹²* wherein R¹¹ and R¹² are
each independently a hydrogen
atom, an alkyl group, an alkoxy
group, an aryl, or an amine
substituted or not by one or two
C₁₋₁₂alkyl groups.
SiR¹³R¹⁴R¹⁵* wherein R13, R14 and
R15 are each independently a
hydrogen atom, an alkyl group,
an alkoxy group, an aryl, or an
amine substituted or not by one or
two C₁₋₁₂alkyl groups.
OR¹⁶* wherein R¹⁶ is a hydrogen
atom, an alkyl group, an alkoxy
group, an aryl, or an amine
substituted or not by one or two
C₁₋₁₂alkyl groups.
SR¹⁷* wherein R¹⁷ is a hydrogen
atom, an alkyl group, an alkoxy
group, an aryl, or an amine
substituted or not by one or two
C₁₋₁₂alkyl groups.
*chiral element
```

7. Process according to claim 1, wherein an asymmetric carbon is present on a leaving group of said aromatic carboxylic acid derivative and/or on the nucleophile, and the compound of general formula (I) obtained is asymmetric.

8. Process according to claim 1, wherein at least one equivalent of NuM is used for one equivalent of starting carboxylic acid derivative.

9. Process according to claim 1, wherein at least one equivalent of a metallic base, preferably butyllithium, sodium hydride, potassium hydride or lithium hydride is used for an equivalent of starting aromatic carboxylic acid derivative in order to form the metal salt corresponding to the acid function of the aromatic carboxylic acid derivative, and at least one equivalent of NuM is added per leaving group of starting molecule to be substituted.

10. Process according to claim 2, wherein NuM is such that M is Li, Mg, Cu, Zn, or MgX wherein X is halogen or alkoxy, and Nu is as described below:
```
Nu
Alkyl, preferably CH₃ or
C₂H₅
Alkenyl, optionally
substituted
Alkynyl optionally
substituted
Aryl optionally
substituted
s-Bu
t-Bu
n-Bu
4-MeOC₆H₄
2-MeOC₆H₄
2,5-diMeC₆H₄
4-Me₂NC₆H₄

2-MeC₆H₄





  wherein Y is O, N or S
  wherein Y is O, N or S
P(Aryl)₂,
PArylAlkyl
O(C₁₋₆alkyl)
S(C₁₋₆alkyl)
  wherein R¹⁸ is a hydrogen atom, an alkyl group, an alkoxy group, an aryl, or an amine substituted or not by one or two C₁₋₁₂alkyl groups
```

11. Process according to claim 2, wherein NuM is such that M is Li, Mg, Cu, Zn, or MgX wherein X is a halogen or an alkoxy and Nu is N(C₁₋₆alkyl)₂, NH(C₁₋₆alkyl), NEt₂, N(CH₂CH₂)₂NMe, NMeBn, NBn₂, NMePh, NHt-Bu NPh₂.

12. Process according to claim 2, wherein NuM is such that M is Li, Mg, Cu, Zn, or MgX wherein X is halogen or alkoxy, and Nu is as described below:
Nu
N(C₁₋₆alkyl)₂
NH(C₁₋₆alkyl),
in particular
NH(tBu)
NEt₂
N(iPr)₂
N(CH₂CH₂)₂NMe
NMeBn
NBn₂
NMePh
NHt-Bu
NPh₂

13. Process according to claim 2, wherein NuM is such that M is Li, Mg, and Nu is as described below:
```
Nu














  wherein Y is O, S or N
  wherein Y is O, S or N
  wherein Y is O, S or N
  wherein Y is O, S or N
NR¹¹R¹²* wherein R¹¹ and R¹² are
each independently a hydrogen
atom, an alkyl group, an alkoxy
group, an aryl, or an amine
substituted or not by one or two
C₁₋₁₂alkyl groups.
SiR¹³R¹⁴R¹⁵* wherein R13, R14 and
R15 are each independently a
hydrogen atom, an alkyl group,
an alkoxy group, an aryl, or an
amine substituted or not by one
or two C₁₋₁₂alkyl groups.
OR¹⁶* wherein R¹⁶ is a hydrogen
atom, an alkyl group, an alkoxy
group, an aryl, or an amine
substituted or not by one or two
C₁₋₁₂alkyl groups.
SR¹⁷* wherein R¹⁷ is a hydrogen
atom, an alkyl group, an alkoxy
group, an aryl, or an amine
substituted or not by one or two
C₁₋₁₂alkyl groups.
*chiral element
```

14. Process according to claim 2, wherein an asymmetric carbon is present on a leaving group of said aromatic carboxylic acid derivative and/or on the nucleophile, and the compound of general formula (I) obtained is asymmetric.

15. Process according to claim 2, wherein at least one equivalent of NuM is used for one equivalent of starting carboxylic acid derivative.

16. Process according to claim 2, wherein at least one equivalent of a metallic base, preferably butyllithium, sodium hydride, potassium hydride or lithium hydride is used for an equivalent of starting aromatic carboxylic acid derivative in order to form the metal salt corresponding to the acid function of the aromatic carboxylic acid derivative, and at least one equivalent of NuM is added per leaving group of starting molecule to be substituted.
